(19) United States
(12) Patent Application Publication
(10) Pub. No.: US 2017/0273652 A1
(43) Pub. Date: Sep. 28, 2017

(54) IMAGE PROCESSING DEVICE, RADIOGRAPHIC IMAGING SYSTEM, IMAGE PROCESSING METHOD, AND NON-TRANSITORY COMPUTER READABLE MEDIUM

(71) Applicant: FUJIFILM CORPORATION, Tokyo (JP)

(72) Inventor: Takashi TAJIMA, Kanagawa (JP)

(21) Appl. No.: 15/384,317

(22) Filed: Dec. 20, 2016

(30) Foreign Application Priority Data
Mar. 22, 2016 (JP) 2016-057639

(51) Int. Cl. A61B 6/00

(52) U.S. Cl. CPC A61B 6/5229; A61B 6/461; A61B 6/5205; A61B 6/4266; A61B 6/505

(57) ABSTRACT

The present disclosure provides an image processing device including: a radiographic image acquisition section that acquires a radiographic image, the radiographic image generated by stitching together radiographic images of an imaging subject imaged by plural radiation detectors, each of which includes a detection face for detecting radiation; a purpose acquisition section that acquires information indicating an interpreting purpose; and an adding section that adds a predetermined assist line to the radiographic image, on the basis of at least one of a reference object corresponding to the interpreting purpose in the radiographic image acquired by the radiographic image acquisition section or the information indicating the interpreting purpose.

### CROSS-REFERENCE TO RELATED APPLICATION

This application claims priority under 35 USC 119 from Japanese Patent Application No. 2016-057639, filed on Mar. 22, 2016, the disclosure of which is incorporated by reference herein.

### BACKGROUND

Technical Field

The present disclosure relates to an image processing device, a radiographic imaging system, an image processing method, and a non-transitory computer readable medium storing an image processing program.

Related Art

Radiographic imaging systems are known in which radiographic imaging of an imaging subject is performed by a radiographic imaging device detecting radiation that has been emitted from a radiation irradiating device and has passed through the imaging subject using a radiation detector.

In such radiographic imaging systems, assist lines are sometimes added to a radiographic image in order to assist interpretation of a radiographic image by a doctor.

Technology to add assist lines to a radiographic image is known in which, for example, a plate for adding assist lines, formed with a metal grid form pattern, is provided between the imaging subject and the radiographic imaging device, and the grid form pattern is imprinted onto the radiographic image as a ghost image.

Alternatively, for example, technology is also known in which grid form assist lines generated by executing software processing or the like are added to and displayed on a radiographic image instead of employing an actual plate for adding assist lines. As this type of technology, Japanese Patent Application Laid-Open (JP-A) No. 2013-135832 describes technology in which a radiographic image is rotated according to an angle of incline of the aorta in the radiographic image, such that the direction of the aorta runs vertically or along a specific direction according to procedure, after which grid lines are superimposed and displayed on the radiographic image.

However, in actual plates for adding assist lines, for example, grid formed of grooves are made in an acrylic base, and a material such as metal with strong radiation shielding properties is provided inside the formed grooves. Thus, in cases in which an actual plate for adding assist lines is employed in imaging, the plate for adding assist lines has a heavy weight, and so sometimes cannot be easily installed. It is also not easy to install the plate for adding assist lines in a position where the assist lines are imprinted as a ghost image in a suitable position on the radiographic image. Thus, in cases in which an actual plate for adding assist lines is employed in imaging, there is a burden on an operator such as a doctor or a technician. It is therefore desirable to improve the work procedure of the operator.

In a case in which a long-length imaging, imaging a large and long imaging subject such as the spine or the lower limbs, is performed, a radiographic image of the entire imaging subject is sometimes imaged by stitching together radiographic images respectively imaged by plural radiation detectors. If an actual plate for adding assist lines were employed for imaging in such cases, there would be a high possibility of artifacts being generated in a section of the image with the assist lines obtained using the plate for adding assist lines in the stitch correction process when stitching the radiographic images imaged by the respective radiation detectors together.

In cases in which assist lines are added without employing an actual plate for adding assist lines, such artifacts are not generated. In the technology described in JP-A No. 2013-135832, the radiographic image is rotated. However, since a radiographic image obtained by the above-described long-length imaging is large, it may be difficult to rotate the radiographic image. Thus, it may be difficult to display assist lines in a suitable position on the radiographic image in the technology described in JP-A No. 2013-135832.

### SUMMARY

An object of the present disclosure is to provide an image processing device, a radiographic imaging system, an image processing method, and a non-transitory computer readable medium that may display an assist line at a suitable position on a radiographic image.

A first aspect of the present disclosure is an image processing device including: a radiographic image acquisition section that acquires a radiographic image, the radiographic image generated by stitching together radiographic images of an imaging subject imaged by plural radiation detectors, each of which includes a detection face for detecting radiation; a purpose acquisition section that acquires information indicating an interpreting purpose; and an adding section that adds a predetermined assist line to the radiographic image, on the basis of at least one of a reference object corresponding to the interpreting purpose in the radiographic image acquired by the radiographic image acquisition section or the information indicating the interpreting purpose.

A second aspect, in the above first aspect, at least one type of the reference object may be predetermined for each interpreting purpose.

A third aspect, in the above aspects, the reference object may be a bone of the imaging subject; and the adding section may extract an image of the reference object from the radiographic image, and may add to the radiographic image an assist line, on the basis of at least one of an image of the reference object or the information indicating the interpreting purpose.

A fourth aspect, in the above aspects, may further include a receiving section that receives designation of the reference object, and, in cases in which the receiving section has received designation of the reference object, the adding section may add to the radiographic image an assist line, on the basis of at least one of the reference object corresponding to the received designation or the information indicating the interpreting purpose.

A fifth aspect, in the above aspects, in cases in which the assist lines are assist lines in which plural lines are combined in grid form, the adding section may rotate the grid form assist lines by an angle derived using the reference object corresponding to the interpreting purpose and the information indicating the interpreting purpose, and may add the rotated assist lines to the radiographic image.

A sixth aspect, in the above aspects, in cases in which an assist line is added to a plurality of radiographic images, the adding section uses the same basis as the basis for adding the assist line to the one radiographic image to add an assist line to another radiographic image from out of the plurality of radiographic images.

A seventh aspect, in the above aspects, the adding section may only add the assist line to an image of an object of interest determined by the interpreting purpose in the radiographic image.

An eight aspect of the present disclosure is a radiographic imaging system including: a radiographic imaging device that includes plural radiation detectors disposed such that detection faces of the plural radiation detectors are in an arranged state, and includes an imaging face having a wider range than one of the detection faces; and the image processing device according to the first aspect, which adds an image of an assist line to a radiographic image imaged by the radiographic imaging device.

An ninth aspect, in the above eighth aspect, may further include a display device that displays the radiographic image to which the image of the assist line has been added by the image processing device.

A tenth aspect of the present disclosure is an image processing method including: acquiring a radiographic image and information indicating an interpreting purpose, the radiographic image generated by stitching together radiographic images of an imaging subject imaged by plural radiation detectors, each of which includes a detection face for detecting radiation; and adding a predetermined assist line to the radiographic image based on at least one of a reference object corresponding to the interpreting purpose in the acquired radiographic image or the information indicating the interpreting purpose.

An eleventh aspect of the present disclosure is a non-transitory computer readable medium storing a program causing a computer to execute a process for image processing, the process including: acquiring a radiographic image and information indicating an interpreting purpose, the radiographic image generated by stitching together radiographic images of an imaging subject imaged by plural radiation detectors, each of which includes a detection face for detecting radiation; and adding a predetermined assist line to the radiographic image, based on at least one of a reference object corresponding to the interpreting purpose in the acquired radiographic image or the information indicating the interpreting purpose.

According to the above aspects, the present disclosure provides an image processing device, a radiographic imaging system, an image processing method, and a non-transitory computer readable medium that may display an assist line at a suitable position on a radiographic image.

### BRIEF DESCRIPTION OF THE DRAWINGS

Exemplary embodiments of the present invention will be described in detail based on the following figures, wherein:

FIG. 1 is a diagram illustrating a configuration of a radiographic imaging system of an exemplary embodiment;

FIG. 2 is a block diagram illustrating a configuration of a radiographic imaging system of the present exemplary embodiment;

FIG. 3 is a schematic view illustrating an example of assist line adding information of the present exemplary embodiment;

FIG. 4 is a flowchart illustrating a flow of an imaging operation of a radiographic image by a radiographic imaging system of the present exemplary embodiment;

FIG. 5 is a flowchart illustrating a flow of image generation and display processing executed by a console of the present exemplary embodiment;

FIG. 6 is an example view illustrating a case in which assist lines have been added to a radiographic image obtained by imaging the entire spine of an imaging subject in a first example;

FIG. 7 is an example view illustrating a case in which assist lines have been added to a radiographic image obtained by imaging the entire spine of an imaging subject in a second example;

FIG. 8 is an example view illustrating a case in which assist lines have been added to a radiographic image obtained by imaging the hip joint of an imaging subject in a third example;

FIG. 9 is an example view illustrating a case in which assist lines have been added to a radiographic image obtained by imaging the front of the entire lower limbs of an imaging subject in a fourth example;

FIG. 10 is an example view illustrating a case in which assist lines have been added to a radiographic image obtained by imaging the side of the lumbar vertebrae of an imaging subject in a fifth example;

FIG. 11 is an example view illustrating a case in which assist lines have been added to a radiographic image obtained by imaging the side of the lumbar vertebrae of an imaging subject after an operation in the fifth example;

FIG. 12 is an example view illustrating a case in which assist lines have been added to a radiographic image obtained by imaging the side of the lumbar vertebrae of an imaging subject in a sixth example; and

FIG. 13 is an example view illustrating a case in which assist lines have been added to a radiographic image obtained by imaging the side of the lumbar vertebrae of an imaging subject in a seventh example.

### DETAILED DESCRIPTION

Detailed explanation follows regarding an exemplary embodiment of the present disclosure, with reference to the drawings. Note that the present disclosure is not limited to the present exemplary embodiment.

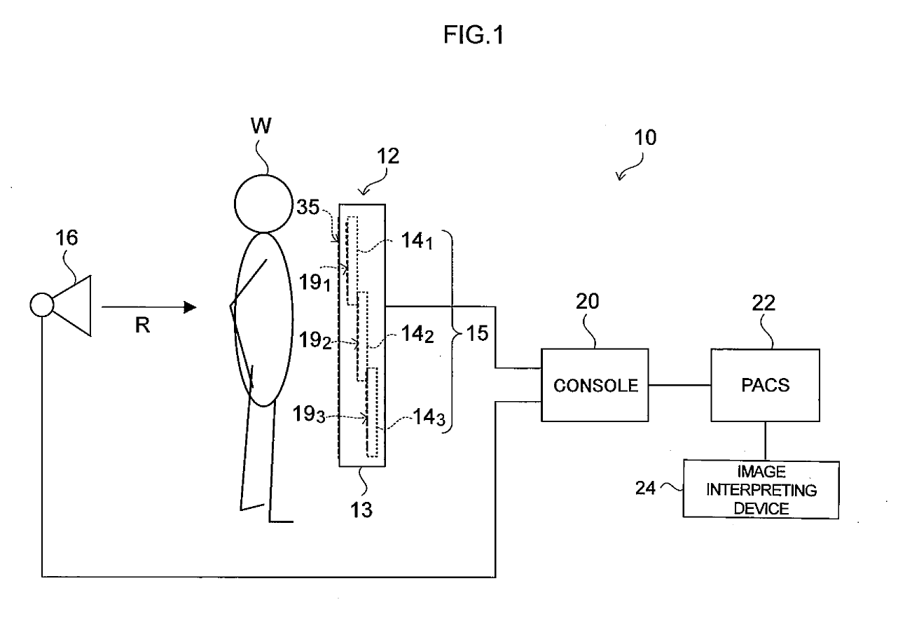

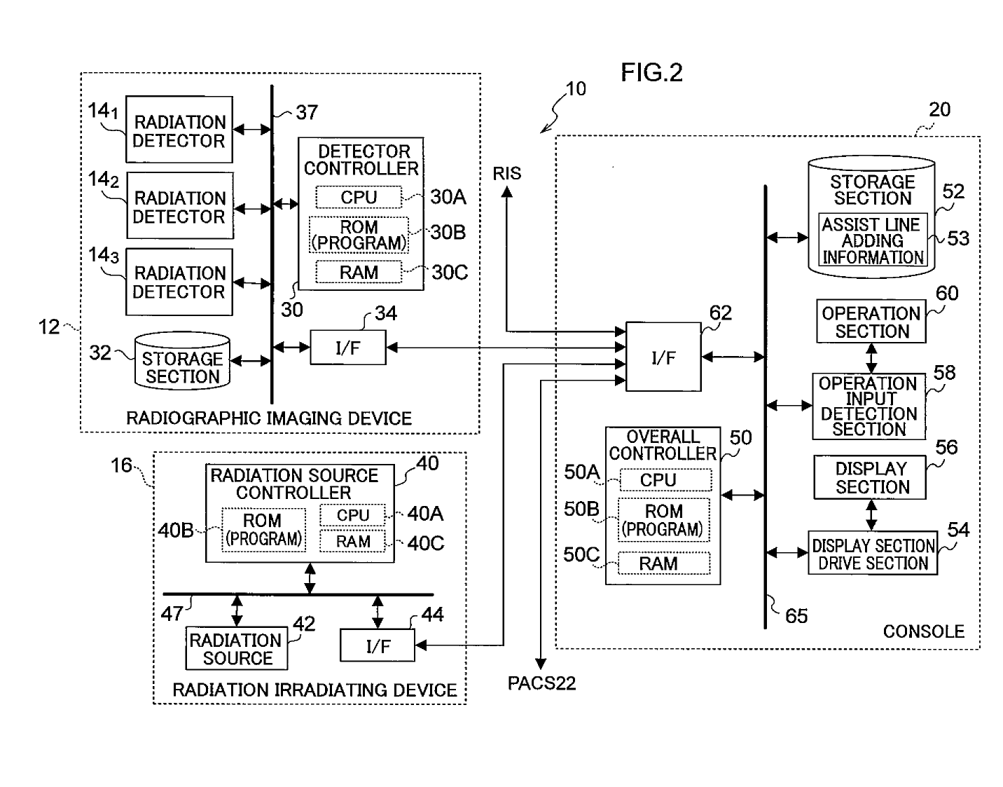

First, explanation follows regarding a configuration of a radiographic imaging system of the present exemplary embodiment, with reference to FIG. 1 and FIG. 2.

As illustrated in FIG. 1, a radiographic imaging system 10 of the present exemplary embodiment includes a radiographic imaging device 12, a radiation irradiating device 16, and a console 20. The radiographic imaging system 10 of the present exemplary embodiment is operated by an operator, such as a doctor or a technician, to perform radiographic imaging based on an imaging menu acquired by the console 20 from an external system such as a radiology information system (RIS), or an imaging menu input by the operator. Note that, in the present exemplary embodiment, the operator refers to a doctor, technician, or the like who performs radiographic imaging using the radiographic imaging device 12.

As illustrated in FIG. 2, the radiation irradiating device 16 of the present exemplary embodiment includes a radiation source controller 40, a radiation source 42, and an interface (I/F) 44. The radiation source controller 40, the radiation source 42, and the I/F 44 are connected together by a bus 47, such as a system bus or a control bus, so as to be capable of exchanging information and the like with each other. The radiation irradiating device 16 irradiates radiation R from the radiation source 42 onto an imaging site (such as the chest region or abdominal region) of an imaging subject W under the control of the console 20.

The radiation source 42 includes a vacuum tube (not illustrated in the drawings) and emits the radiation R under the control of the radiation source controller 40. In the below explanation, the position of the vacuum tube is the same as the position of the radiation source 42.

The radiation source controller 40 controls the radiation source 42 based on radiation conditions of the radiation R, such as tube voltage, tube current, and radiation duration. The radiation source controller 40 includes a central processing unit (CPU) 40A, read only memory (ROM) 40B, and random access memory (RAM) 40C. Programs, etc. to be executed by the CPU 40A are pre-stored in the ROM 40B. The radiation irradiating device 16 performs control relating to the radiation R emitted from the radiation source 42 by the CPU 40A executing a program stored in the ROM 40B. The RAM 40C temporarily stores various data.

The I/F 44 exchanges various information with the console 20 by wireless communication, wired communication, or the like.

Note that the radiation irradiating device 16 may also include an operation input section in order for the operator to manually set the radiation conditions directly to the radiation irradiating device 16, and a display section for displaying the set radiation conditions and so on. In a case in which the radiation conditions have been manually set by the operator, the radiation irradiating device 16 transmits information expressing setting values that have been manually set, the current status (such as a standby state, a preparatory state, an exposure in-progress state, or an exposure complete state), and so on to the console 20.

The radiographic imaging device 12 of the present exemplary embodiment is what is referred to as an digital radiography (DR) cassette employed for long-length imaging. Note that “long-length imaging” refers to imaging large and long site on the imaging subject W, such as the entire spine or the entire lower limbs. “Employed for long-length imaging” refers to being employed for long-length imaging. However, the radiographic imaging device 12 is not dedicated to long-length imaging, and may be employed in imaging that is not long-length imaging, such as of the chest region or head.

As illustrated in the example in FIG. 1, the radiographic imaging device 12 of the present exemplary embodiment includes a radiation detector group 15 inside a casing 13, and the radiation detector group 15 includes three radiation detectors 14₁ to 14₃. When referred to collectively below, the numeral suffix denoting each individual detector is omitted, and the radiation detectors 14₁ to 14₃ are referred to as “radiation detectors 14”. Note that the number of radiation detectors 14 is not limited to the number in the present exemplary embodiment.

In a case in which radiographic imaging are performed, radiographic imaging is performed by all the radiation detectors 14 by emitting radiation R a single time (what is referred to as one shot).

The radiation detectors 14 of the present exemplary embodiment are disposed in a state in which detection faces 19₁ to 19₃ (regions of pixels (not illustrated in the drawings) effective for imaging) face the imaging subject W. When referred to collectively below, the numeral suffix denoting each detection face is omitted, and the detection faces 19₁ to 19₃ are referred to as “detection faces 19”. Note that, as illustrated in FIG. 1, an end portion of each radiation detector 14 is disposed overlapping an end portion of an adjacent radiation detector 14 in the radiographic imaging device 12 of the present exemplary embodiment.

In a case in which there is a spacing separating the end portions of adjacent radiation detectors 14, there may be cases in which parts of the imaging site of the imaging subject W are not imaged. Further, it is also difficult to dispose the end portions of adjacent radiation detectors 14 in close contact with each other without any gaps therebetween, due to manufacturing variations in the radiation detectors 14. Thus, as illustrated in FIG. 1, in the present exemplary embodiment, the end portion of the detection face 19 of a radiation detector 14 and the end portion of the detection face 19 of an adjacent radiation detector 14 are disposed overlapping each other in the radiation detector group 15. Specifically, the detection faces 19 at the end portions of adjacent radiation detectors 14 are overlapped in the direction of incidence of the radiation R. Note that the range (size) of the detection face 19 at overlapping portions where the radiation detectors 14 overlap each other is predetermined according to the degree at which the radiation R irradiated from the radiation irradiating device 16 is obliquely incident, and so on.

Since the radiographic imaging device 12 of the present exemplary embodiment includes the plural radiation detectors 14 disposed in the above manner, the whole of the radiographic imaging device 12 has a detection face that is longer than the detection face 19 of just one radiation detector 14.

Radiation R that has passed through the imaging subject W is irradiated onto the radiation detectors 14 of the radiographic imaging device 12. Each radiation detector 14 of the radiographic imaging device 12 generates charges according to the amount of radiation R that has passed through the imaging subject W, and generates and outputs image data of a radiographic image (hereafter simply referred to as “radiographic image”) based on the amount of generated charges.

As illustrated in FIG. 2, the radiographic imaging device 12 of the present exemplary embodiment also includes a detector controller 30, a storage section 32, and an I/F 34. The detector controller 30, the radiation detectors 14₁ to 14₃, the storage section 32, and the I/F 34 are connected together by a bus 37, such as a system bus or a control bus, so as to be capable of exchanging information and the like with each other.

The detector controller 30 controls the radiographic imaging device 12 as a whole. As illustrated in FIG. 2, the detector controller 30 of the present exemplary embodiment includes a CPU 30A, ROM 30B, and RAM 30C. Programs, etc. to be executed by the CPU 30A are pre-stored in the ROM 30B. The radiographic imaging device 12 controls the radiation detectors 14 by the CPU 30A executing a program stored in the ROM 30B.

The storage section 32 stores radiographic images and the like imaged by the radiation detectors 14.

The I/F 34 exchanges various information with the console 20 by wireless communication, wired communication, or the like.

In the present exemplary embodiment, radiographic images imaged by the radiographic imaging device 12 are input to the console 20 through the I/F 34.

The console 20 of the present exemplary embodiment is a server computer. As illustrated in FIG. 2, the console 20 of the present exemplary embodiment includes an overall controller 50, a storage section 52, a display section drive section 54, a display section 56, an operation input detection section 58, an operation section 60, and an I/F 62. The overall controller 50, the storage section 52, the display section drive section 54, the display section 56, the operation input detection section 58, the operation section 60, and the I/F 62 are connected together by a bus 65, such as a system bus or a control bus, so as to be capable of exchanging information and the like with each other. In the present exemplary embodiment, the console 20 functions as an image processing device of the present disclosure.

The console 20 transmits at least one from out of radiographic images that have been image-processed (described in detail later) by the overall controller 50, or radiographic images (prior to image processing) as acquired from the radiographic imaging device 12, to a picture archiving and communication system (PACS) 22. The PACS 22 manages radiographic images received from the console 20. The radiographic images managed by the PACS 22 are displayed on at least one out of a display section (not illustrated in the drawings) of an image interpreting device 24 or the display section 56 of the console 20, according to an instruction from a doctor how interprets the radiographic images, for example. Note that the image interpreting device 24 is a device employed in order to interpret the imaged radiographic images by a doctor. There is no particular limitation thereto, and examples include what is referred to as a viewer, as well as personal computers and personal digital assistants (PDAs), such as tablet terminals and smartphones, employed by a doctor. Note that “interpreting” in the present exemplary embodiment is not limited to cases in which a diagnosis is made, and also includes cases in which an affected area, a site of interest, or the like is observed by radiographic imaging.

The overall controller 50 controls overall operation of the console 20. As illustrated in FIG. 2, the overall controller 50 includes a CPU 50A, ROM 50B, and RAM 50C. Various programs, etc. to be executed by the CPU 50A are pre-stored in the ROM 50B. The RAM 50C temporarily stores various data.

The overall controller 50 of the console 20 of the present exemplary embodiment controls the radiographic imaging device 12 and the radiation irradiating device 16 using an imaging menu and various other information acquired, for example, from an external system by wireless communication. The overall controller 50 of the console 20 performs plural types of predetermined image processing on radiographic images acquired from the radiographic imaging device 12. Explanation follows regarding the image processing executed by the overall controller 50 of the present exemplary embodiment.

As described above, the radiographic imaging device 12 of the present exemplary embodiment performs imaging using the radiation detectors 14 which are overlapped in what is referred to as a stepped pattern. Radiographic imaging is performed by each of the three radiation detectors 14, and so the overall controller 50 performs image processing by stitching together the radiographic images imaged by each radiation detector 14 (hereafter referred to as “stitching processing”) to obtain a long-length radiographic image using the whole of the radiographic imaging device 12.

For overlapping radiation detectors 14, a shadow of the radiation detector 14 nearer to the radiation irradiating device 16 may be imprinted as an image of the step onto the radiographic image imaged by the radiation detector 14 further from the radiation irradiating device 16. For example, in the case illustrated in FIG. 1, an end portion of the radiation detector 14₁ may be imprinted as a step image onto the radiographic image imaged by the radiation detector 14₂, and an end portion of the radiation detector 14₂ may be imprinted as a step image onto the radiographic image imaged by the radiation detector 14₃. The overall controller 50 therefore performs image processing to remove the step image from each radiographic image (hereafter referred to as “step removal processing”).

Note that there is no particular limitation to the respective image processing methods for stitching processing and step removal processing. For example, step removal processing may be performed in the below manner.

A step image is included in the radiographic image imaged by the respective radiation detector 14 that is further from the radiation irradiating device 16, and so the step removal processing of the present exemplary embodiment is performed on the radiographic image imaged by the respective radiation detector 14 further from the radiation irradiating device 16. There is no particular limitation to the method by which the overall controller 50 ascertains whether a radiographic image acquired from the radiation detector group 15 has been imaged by a radiation detector 14 further from the radiation irradiating device 16, or by a radiation detector 14 nearer thereto. For example, information indicating whether each radiation detector 14 is a radiation detector 14 further away or nearer than an adjacent radiation detector 14 may be added to the radiographic image and output to the console 20.

When correcting a step image, the overall controller 50 first detects the position of the step image in the radiographic image. There is no particular limitation to the detection method of the position of the step image. As a specific example, the overall controller 50 of the present exemplary embodiment detects the position of a boundary between the step image and the normal image by detecting an image expressing a straight line in the radiographic image, and detects the position of the step image based on the detected position of the boundary. Note that the boundary between the step image and the normal image is simply referred to below as “boundary”. There is no particular limitation to the method of detecting a straight line, and a general method, such as the Hough transform, may be employed. There is also no particular limitation to the method of detecting the position of a step image from the position of the boundary, and, for example, the region between the position of the boundary and a specific end portion of the radiographic image may be detected as the step image.

When detecting the position of the boundary in the radiographic image, processing to detect the position of the boundary may be performed on the entire radiographic image, or detection of the position of the boundary may be performed by searching within a search range, this search range being a region in which the position of the boundary is estimated to be contained. For example, a possible range within which the position of the step image (the position of the boundary) may lie in the radiographic image may be obtained based on the design or by experimentation, and this range applied as the search range. Detecting the position of the boundary within the estimated search range enables the detection accuracy to be improved and also enables the detection duration to be reduced, compared to cases in which the position of the boundary is detected from the entire radiographic image.

When the position of the step image is detected, the overall controller 50 then corrects the step image included in the radiographic image. The overall controller 50 of the present exemplary embodiment corrects the step image by correcting to reduce a difference in density between the density of the step image and the density of the normal image. Note that the step correction can be more accurately performed by performing offset correction, gain correction, pixel defect correction, and the like on the radiographic image prior to correcting to reduce the difference in density.

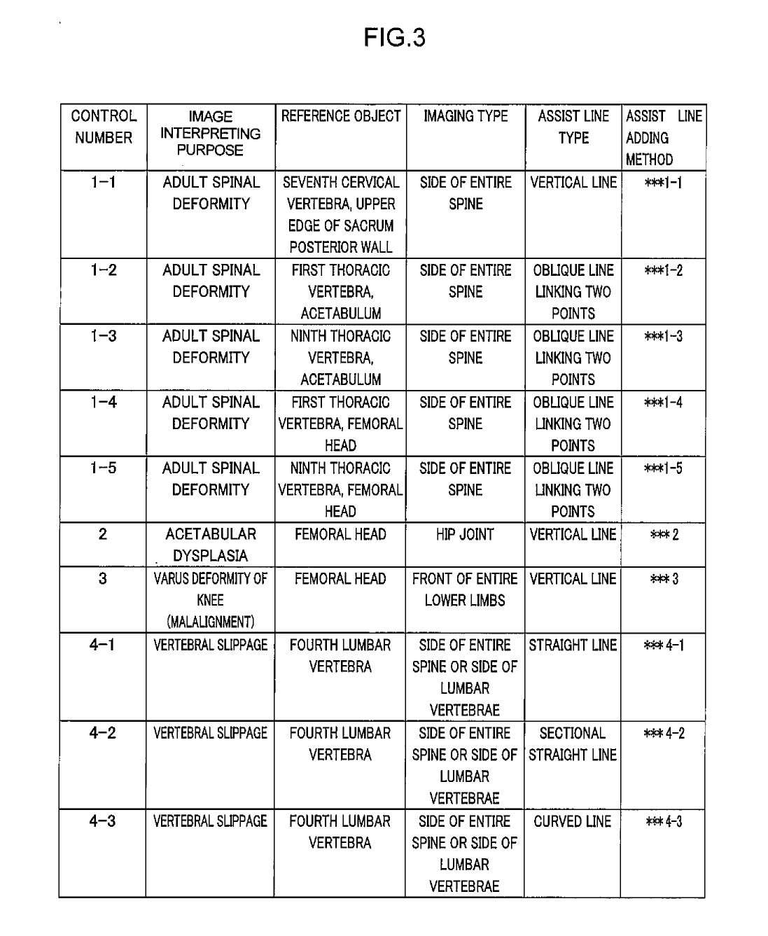

The overall controller 50 of the present exemplary embodiment also performs image processing (described in detail later) to add to the radiographic image an image of assist lines suitable for the purpose of interpreting by a doctor (hereafter referred to as “interpreting purpose”), based on assist line adding information 53 (see FIG. 3) pre-stored in the storage section 52. In the present exemplary embodiment, the method by which the overall controller 50 adds assist lines to the radiographic image differs according to the interpreting purpose, a reference object, and the type of assist line. In the present exemplary embodiment, the “interpreting purpose” includes observation of alignment of the bones of the imaging subject W, observation of the affected area according to what kind of case the imaging subject W is, and so on. The “reference object” is an object of reference for the position and so on, of the assist lines to be added, which may be different from the affected area or site of interest that the doctor wants to observe. The “assist line type” is the type of assist line to be added to the radiographic image, described in detail later, which may be a vertical line, an oblique line, or a curved line, for example.

Note that in the present exemplary embodiment, there is no particular limitation to the specific manner in which assist lines are “added” to the radiographic image. For example, assist lines may be added by being drawn onto the radiographic image itself, or assist lines may be added by superimposing an image of the assist lines onto the radiographic image (by superimposing an assist line image layer onto a radiographic image layer, for example).

The display section drive section 54 illustrated in FIG. 2 controls the display of various information on the display section 56. The display section 56 displays an imaging menu, radiographic images that have been imaged, and so on. The operation input detection section 58 detects an operation state of the operation section 60 by the operator or the like. The operation section 60 is used by a doctor to input instruction operations for performing radiographic imaging, instructions relating to image processing of imaged radiographic images, and so on. The operation section 60 may take the form of a keyboard, for example, or may take the form of a touch panel that is integrated with the display section 56.

The I/F 62 exchanges various information between the PACS 22 and the RIS by wireless communication or the like. The I/F 62 also exchanges various information between the radiographic imaging device 12 and the radiation irradiating device 16.

The storage section 52 stores and retains radiographic images and various other types of data. Note that the storage section 52 of the present exemplary embodiment stores and retains the assist line adding information 53, an example of which is illustrated in FIG. 3. The assist line adding information 53 is information relating to assist lines for adding to the radiographic image, and, as illustrated in the example in FIG. 3, is information indicating correspondence relationships between interpreting purpose, reference object, imaging type, assist line type, and assist line adding method. Note that, in the present exemplary embodiment, the “assist line adding method” refers to information describing a specific method for adding assist lines, and what is referred to as a sub-program is an example of such information. In cases in which a sub-program is employed as the assist line adding method, the sub-program may be stored in a separate storage location to the storage section 52, and the assist line adding information 53 may associate information indicating the sub-program file name, storage location, and so on to the other information such as the interpreting purpose. In the assist line adding information 53 illustrated in the example in FIG. 3, for example, “***1-1” to “***4-3” indicate sub-program file names. The “imaging type” refers to types of imaging relating to imaging site, such as “imaging of side of entire spine” and “imaging of entire lower limbs”.

Explanation follows regarding an operation when radiographic imaging is performed by the radiographic imaging system 10 of the present exemplary embodiment.

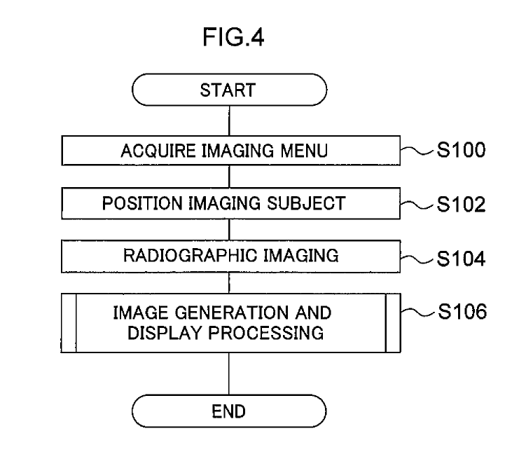

First, explanation follows regarding the overall flow of radiographic imaging performed by the radiographic imaging system 10 of the present exemplary embodiment. FIG. 4 is a flowchart illustrating the overall flow of radiographic imaging performed by the radiographic imaging system 10 of the present exemplary embodiment.

At step S100 in FIG. 4, the overall controller 50 of the console 20 acquires an imaging menu. The imaging menu includes an imaging order from the doctor who requested the imaging, imaging conditions such as the tube voltage of the radiation source 42, the amount of radiation R to be irradiated onto the imaging subject W (the product (mAs value) of tube current (mA) and time (sec)), as well as information relating to the imaging subject W. The overall controller 50 may acquire an imaging menu from an external system through the I/F 62, for example, or may acquire an imaging menu input to the operation section 60 by the operator.

Next, at step S102, the operator positions the imaging subject W.

Next, at step S104, the overall controller 50 of the console 20 emits radiation R from the radiation irradiating device 16 according to the acquired imaging menu, and the radiation R that has passed through the imaging subject W is detected by the radiation detectors 14, such that a radiographic image is imaged by the radiographic imaging device 12.

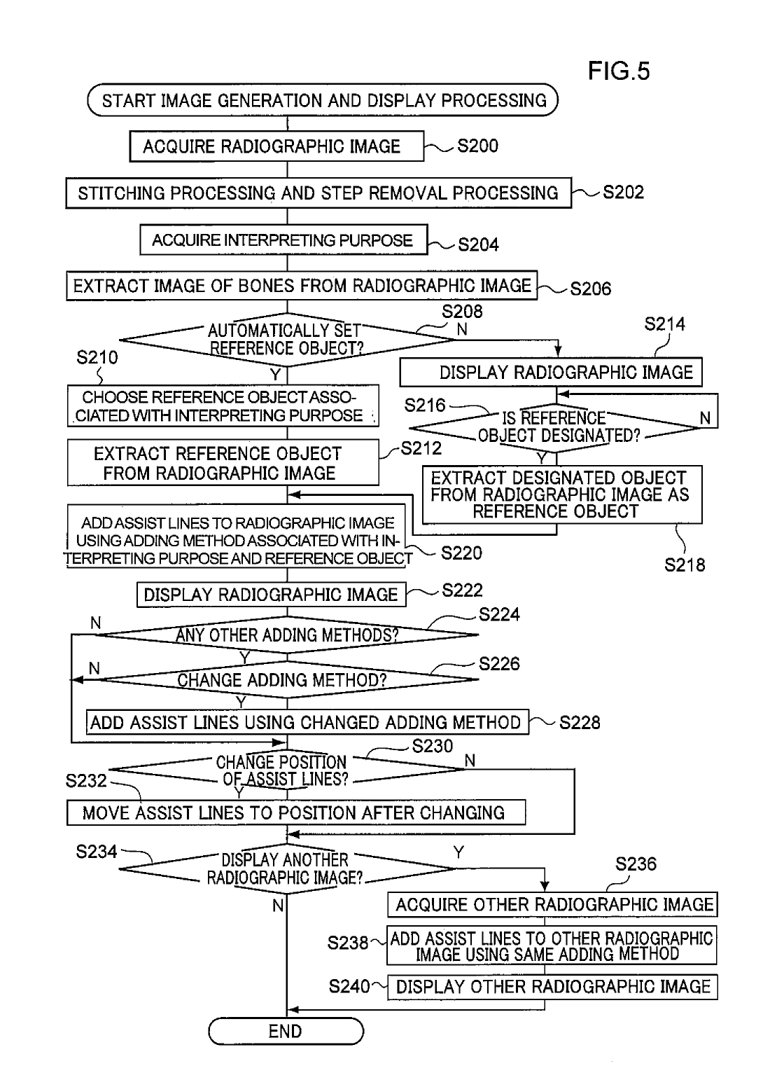

Next, at step S106, the overall controller 50 of the console 20 executes the image generation and display processing illustrated in FIG. 5.

At step S200 illustrated in FIG. 5, the overall controller 50 acquires the radiographic image. Specifically, the overall controller 50 acquires each of the radiographic images imaged by each radiation detector 14 of the radiation detector group 15 from the storage section 52.

Next, at step S202, the overall controller 50 performs the above-described stitching processing and step removal processing. A long-length radiographic image is obtained from the whole of the radiographic imaging device 12 by performing this processing. Note that the radiographic image obtained at this step may be stored in the storage section 52. By storing the radiographic image after stitching processing and step removal processing in the storage section 52 in this manner, the radiographic image after stitching processing and step removal processing can be acquired, thereby enabling the present step to be omitted in a case in which the radiographic image is displayed again.

Next, at step S204, the overall controller 50 acquires the interpreting purpose (information indicating the interpreting purpose). The method by which the overall controller 50 acquires the interpreting purpose may be predetermined, and there is no particular limitation thereto. For example, in cases in which the interpreting purpose is included in the imaging menu, the interpreting purpose may be acquired from the imaging menu acquired at step S100. Alternatively, a interpreting purpose that has been input by the operator through the operation section 60 may be acquired.

Next, at step S206, the overall controller 50 extracts an image of the bones of the imaging subject W from the radiographic image. The method by which the overall controller 50 extracts the image of bones may be predetermined, and there is no particular limitation thereto. For example, reference images of bones corresponding to the age and gender of the imaging subject W, the interpreting purpose, the imaging type, the imaging site, and so on (hereafter referred to as “reference images”) may be pre-stored in the storage section 52, and the image of bones may be extracted from the radiographic image by executing pattern matching using a reference image in which at least one out of the interpreting purpose, the imaging type, or the imaging site match that of the radiographic image.

Next, at step S208, the overall controller 50 determines whether or not to set the reference object automatically. In the console 20 of the present exemplary embodiment, the reference object is normally set automatically, and the reference object is only set manually by the doctor in cases in which an instruction to set the reference object has been received from the doctor through the operation section 60. In cases in which in which an instruction to set the reference object has not been received through the operation section 60 even after a specific duration has elapsed, affirmative determination is made and processing proceeds to step S210.

At step S210, the overall controller 50 chooses a reference object associated with the interpreting purpose acquired at step S204. Specifically, the overall controller 50 chooses a reference object associated with the interpreting purpose with reference to the assist line adding information 53. Note that, in cases in which plural different reference objects are associated with a single interpreting purpose in the assist line adding information 53, the single reference object selected by the overall controller 50 from out of the plural different associated reference objects is chosen as the reference object. For example, in the example illustrated in FIG. 3, the reference object of control number 1-1 is “seventh cervical vertebra, upper edge of the sacrum posterior wall”, the reference object of control number 1-2 is “first thoracic vertebra, acetabulum”, the reference object of control number 1-3 is “ninth thoracic vertebra, acetabulum”, the reference object of control number 1-4 is “first thoracic vertebra, femoral head”, and the reference object of control number 1-5 is “ninth thoracic vertebra, femoral head”, these all having a common interpreting purpose of “adult spinal deformity”. Namely, in this case, five types of reference object are associated with a single interpreting purpose. The overall controller 50 chooses a single reference object selected from these reference objects to be the reference object. There is no particular limitation to the selection method. The console 20 of the present exemplary embodiment selects a reference object associated with a control number indicated in advance by the doctor or the like.

Next, at step S212, the overall controller 50 extracts the image of the reference object chosen in the processing of step S210 from the radiographic image, and processing proceeds to step S220. The method by which the overall controller 50 extracts the image of the reference object may be predetermined, and there is no particular limitation thereto. For example, a similar method to pattern matching, explained as a method to extract an image of bones from the radiographic image in the processing of step S206, may be employed. Alternatively, for example, depending on the reference object, an approximate position in the radiographic image may be identified by using physical features of the imaging subject W, such as age and gender. In cases in which the femoral head has been chosen as the reference object, for example, the center (backbone) of the body of the imaging subject W is often located at a central region of the radiographic image, and an approximate distance from the center to the femoral head can be pre-obtained. In such cases, a position that is separated by the pre-obtained distance from the center of the radiographic image may be set as the position of the femoral head, and an image of the surrounding bones may be extracted as an image of the femoral head.

In cases in which an instruction to set the reference object has been received through the operation section 60 within the specific duration at step S208, negative determination is made, and processing proceeds to step S214.

At step S214, the overall controller 50 displays the radiographic image on the display section 56. In a case in which the radiographic image is displayed on the display section 56, the doctor designates through the operation section 60 a reference object in the radiographic image displayed on the display section 56. Next, at step S216, the overall controller 50 makes a negative determination until the doctor has designated a reference object through the operation section 60, and makes an affirmative determination in a case in which a reference object has been designated. Then, the processing proceeds to step S218.

At step S218, the overall controller 50 extracts as the reference object an image of bones, including the image of the designated object designated by the doctor from the radiographic image. Then, the processing proceeds to step S220.

At step S220, the overall controller 50 adds assist lines to the radiographic image using an adding method associated with the interpreting purpose and reference object obtained by the above-described processing. Specifically, the overall controller 50 refers to the assist line adding information 53, and adds assist lines to the radiographic image using an assist line adding method associated with the interpreting purpose acquired at step S204, and the reference object chosen at step S210 or the reference object extracted at step S218. For example, in a case in which the interpreting purpose is for “acetabular dysplasia” and the reference object is “femoral head”, the overall controller 50 refers to control number 2 in the assist line adding information 53 and executes the sub-program with the file number “***2” as the assist line adding method. Accordingly, assist lines are added to the radiographic image.

Next, at step S222, the overall controller 50 displays the radiographic image to which the assist lines have been added on the display section 56. Note that, in the present exemplary embodiment, an assist line that is a pair formed of a white line and a black line is displayed on the display section 56 as each assist line. Namely, in the present exemplary embodiment, two lines, these being a black and a white line, are treated the same as single line, thereby making the assist lines easier to see in the black and white radiographic image. Note that there is no particular limitation to the color of the assist lines, and another color such as red may be employed, or a mode may employed in which the color can be changed according to an instruction by the doctor.

Next, at step S224, the overall controller 50 determines whether or not there is an assist line adding method associated with the interpreting purpose and the reference object other than the method of adding assist lines used in the processing of step S220, namely, other than the method by which the assist lines have been added to the radiographic image currently displayed on the display section 56. For example, in the example illustrated in FIG. 3, in cases in which assist lines have been added by the assist line adding method associated with control number 2 in the processing of step S220, there is no other assist line adding method, and so negative determination is made, and processing proceeds to step S230. As another example, in the example illustrated in FIG. 3, in cases in which assist lines have been added by the assist line adding method associated with control number 1-1 in the processing of step S220, the assist line adding methods respectively associated with control numbers 1-2 to 1-5 exist as other assist line adding methods, and so affirmative determination is made at the present step, and processing proceeds to step S226. Note that in cases in which affirmative determination is made, the overall controller 50 displays on the display section 56 information indicating there is an adding method other than the method of adding assist lines to the radiographic image that is currently being displayed, and information relating to this other adding method.

At step S226, the overall controller 50 determines whether or not to change the assist line adding method. In cases in which an instruction to change the adding method and instruction relating to the adding method to be changed to has been received through the operation section 60 from the doctor who has confirmed the assist lines added to the radiographic image displayed on the display section 56, affirmative determination is made, and processing proceeds to step S228. At step S228, the overall controller 50 changes the assist lines added to the radiographic image displayed on the display section 56 to the assist lines added by the method instructed by the doctor. Then, the processing proceeds to step S230. At step S226, in cases in which an instruction relating to changing the adding method and the adding method to be changed to has not been received even after a specific duration has elapsed, and in cases in which an instruction not to change the added assist lines (establishing the position of the assist lines) has been received from the user through the operation section 60, negative determination is made, and processing proceeds to step S230.

At step S230, the overall controller 50 determines whether or not to change the position of the assist lines. In the console 20 of the present exemplary embodiment, the position of the assist lines added to the radiographic image can be changed by the doctor. In the console 20 of the present exemplary embodiment, the position of the assist line image on the radiographic image (hereafter simply referred to as “assist line position”) may be changed in a direction parallel to an imaging face 35 of the radiographic imaging device 12. In the console 20 of the present exemplary embodiment, the doctor uses the operation section 60 to instruct the position of the assist lines after changing in order to change the assist line position. For example, an instruction regarding the assist line position after changing may be made using a pointing device such as a mouse as the operation section 60, and moving (what is referred to as dragging and dropping) the assist line image added to the radiographic image displayed on the display section 56 to the desired changed position using the operation section 60 such as the pointing device.

At step S230, affirmative determination is made in cases in which an instruction regarding an assist line position after changing has been received from the doctor, and processing proceeds to step S232.

At step S232, the overall controller 50 moves the assist line image added to the radiographic image displayed on the display section 56 to the assist line position as instructed by the doctor. Then, the processing proceeds to step S234. Specifically, the overall controller 50 derives the movement amount of the assist lines by deducting the assist line position prior to changing from the assist line position after changing received from the doctor, and changes the assist line position according to the derived movement amount.

In cases in which an instruction regarding the assist line position after changing has not been received through the operation section 60 even after a specific duration has elapsed at step S230, negative determination is made and processing proceeds to step S234.

At step S234, the overall controller 50 determines whether or not to display another radiographic image. For example, affirmative determination is made in cases in which a radiographic image other than the radiographic image currently being displayed on the display section 56 is to be displayed on the display section 56, such as when comparing the current radiographic image of the imaging subject W to a radiographic image of the same imaging subject W that has been imaged in the past in order to observe changes in the affected area, when comparing radiographic images of the affected area before and after an operation, and when comparing a radiographic image imaged from the front to a radiographic image imaged from the side of the imaging subject W. Processing then proceeds to step S236. Note that whether or not to display other radiographic images on the display section 56 may be designated by the doctor through the operation section 60, alternatively, whether or not to display plural radiographic images on the display section 56 may be set in advance by the doctor or the like.

At step S236, the overall controller 50 acquires from the storage section 52 the other radiographic image to display on the display section 56. Note that in cases in which a radiographic image on which stitching processing and step removal processing has already been performed is acquired from the storage section 52, the acquired radiographic image is displayed on the display section 56. However, in cases in which radiographic images imaged by the respective radiation detectors 14 of the radiation detector group 15 are acquired, stitching processing and step removal processing, executed by the processing of step S202, is performed on the acquired radiographic images.

Next, at step S238, the overall controller 50 adds assist lines to the radiographic image acquired at step S236 using the same adding method as that used to add assist lines to the radiographic image displayed on the display section 56. Specifically, the overall controller 50 of the present exemplary embodiment refers to the assist line adding information 53, and adds assist lines to the radiographic image acquired in the processing of step S236 using the reference object and the assist line adding method employed when adding the assist lines added to the radiographic image displayed on the display section 56.

Next, at step S240, the overall controller 50 displays on the display section 56 the other radiographic image to which the assist lines have been added. Then, the present image generation and display processing is ended. Note that in the present exemplary embodiment, when displaying the other radiographic image to which the assist lines have been added, the radiographic image to which assist lines have been added that was displayed in the processing of step S222 is displayed alongside.

In cases in which another radiographic image is not displayed at step S234, negative determination is made, and the present image generation and display processing is ended. Note that the overall controller 50 may store in the storage section 52 the radiographic image in a state in which the assist lines have been added.

After completing the image generation and display processing in this manner, the overall operation of radiographic imaging is also ended.

Explanation follows regarding specific examples of adding assist lines according to the interpreting purpose using the processing of step S220 and step S238 of the image generation and display processing executed by the console 20 of the present exemplary embodiment.

### Example 1

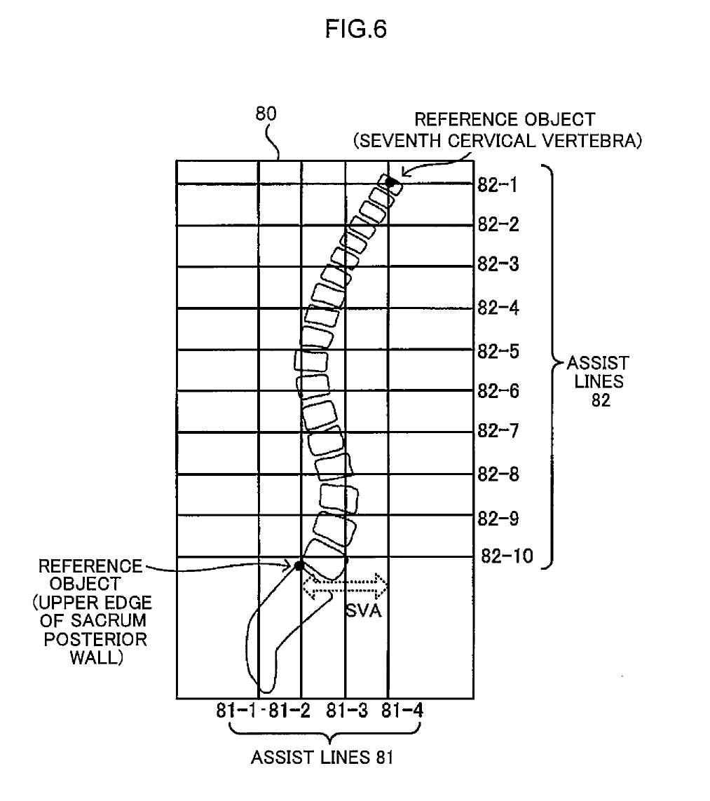

Explanation first follows regarding a case in which the interpreting purpose is for “adult spinal deformity” and the reference object is the “seventh cervical vertebra, upper edge of the sacrum posterior wall”. According to the assist line adding information 53 illustrated in the example in FIG. 3, in such a case, a control number is 1-1, the imaging type is “side of entire spine”, the assist line type is “vertical line”, and the assist line adding method is the method of the sub-program with the file name “***1-1”. FIG. 6 is an example of a schematic drawing in a case in which assist lines have been added to a radiographic image 80 obtained by imaging the entire spine of the imaging subject Win the present example.

The sagittal vertical axis (SVA) generally serves as an index in diagnosing adult spinal deformity. An SVA of less than 40 mm is a normal value. Note that, as illustrated in FIG. 6, the SVA is the distance in the horizontal direction from the vertical line (the line going downward in the direction of gravity) of the seventh cervical vertebra to the upper edge of the sacrum posterior wall. Thus, in the present example, the overall controller 50 adds assist lines to the radiographic image in order to make the SVA clearer for the doctor. In the example illustrated in FIG. 6, an assist line 81-4, this being a vertical line that passes through the center of the seventh cervical vertebra, is important in diagnosing adult spinal deformity, and so the assist line 81-4 is an essential assist line. Thus, the overall controller 50 adds other assist lines 81 that are parallel to and have the assist line 81-4 as a reference. Although the manner in which the other assist lines 81 is added is optional, an assist line 81-2 that passes through the upper edge of the sacrum posterior wall is preferably added. The other assist line 81 (the assist line 81-3 in the example illustrated in FIG. 6) between the assist lines 81-2 and 81-4 and the assist line 81-1 are optional, and do not need to be added. However, when added, the spacing between the parallel assist lines is preferably a uniform spacing, and there is preferably an actual spacing of 3 cm to 5 cm therebetween to assist diagnosis and so on by the doctor.

In the example illustrated in FIG. 6, the overall controller 50 also adds to the radiographic image 80 plural parallel assist lines 82 (82-1 to 82-10) that intersect (are orthogonal to in the example illustrated in FIG. 6) the assist lines 81. There is no particular limitation to the spacings between the respective assist lines 82, which may be uniform spacings, or may be non-uniform spacings. In cases in which the spacings between the assist lines 82 are uniform, for example, these spacings may be the same as the spacings between the assist lines 81, or may be spacings that suit the size of a specific vertebra (a vertebra with a representative size). Alternatively, assist lines may be added at specific spacings between the assist line 82 passing through the seventh cervical vertebra and the assist line 82 passing through the upper edge of the sacrum posterior wall, or a specific number of assist lines may be added therebetween. In cases in which the spacings between the respective assist lines 82 are non-uniform, for example, assist lines 82 may be added that pass through the center of each vertebra, or the spacings between the assist lines 82 may be set to match the spacings between each vertebra.

According to the above, a case in which the overall controller 50 adds the assist lines 81 and the assist lines 82 to the radiographic image 80 separately, has been explained. However, the assist line adding method is not limited thereto. For example, assist lines in a grid form formed of plural pre-combined lines may be superimposed on the radiographic image 80. In such a case, the overall controller 50 may add the grid form assist lines with the position of the assist line 81-4 as a reference, and may also adopt a mode in which the spacing of the grid is adjusted so as to add an assist line at the position of the assist line 81-2 in cases in which the spacings in the grid are variable.

The overall controller 50 may also derive the SVA from the radiographic image 80 and display the derived SVA on the display section 56.

### Example 2

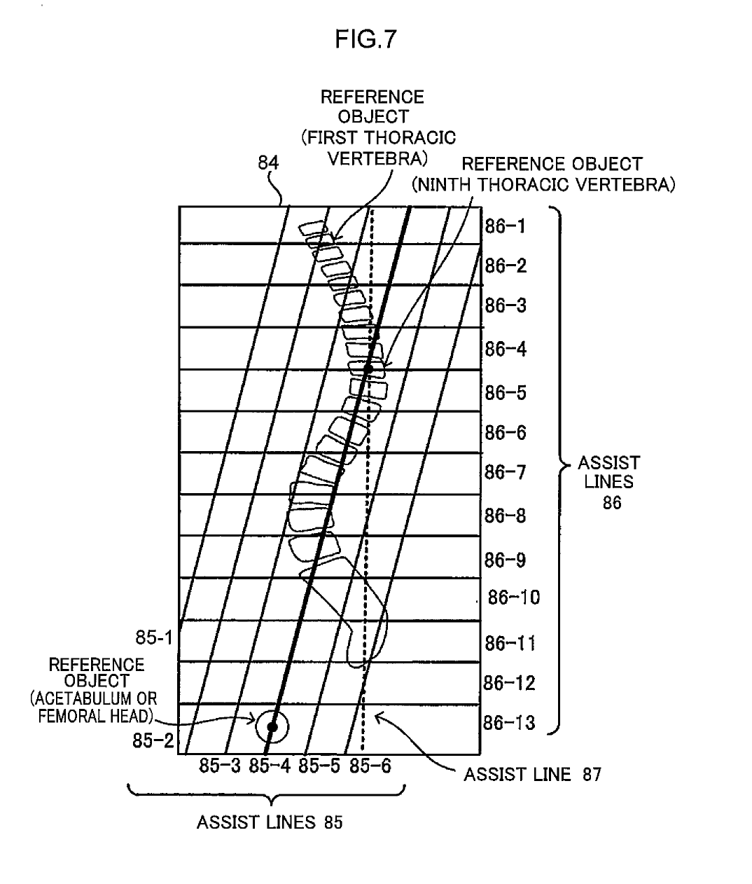

Explanation follows regarding a case in which the interpreting purpose is for “adult spinal deformity”, and the reference object is “ninth thoracic vertebra, femoral head”. According to the assist line adding information 53 illustrated in the example in FIG. 3, in such a case, the control number is 1-5, the imaging type is “side of entire spine”, the assist line type is “oblique line linking two points”, and the assist line adding method is the method of the sub-program with the file name “***1-5”. FIG. 7 is an example of a schematic drawing in a case in which assist lines have been added to a radiographic image 84 obtained by imaging the entire spine of the imaging subject Win the present example.

The degree of curvature of the spine is generally observed to diagnose adult spinal deformity. Thus, in the present example, the overall controller 50 adds assist lines to the radiographic image in order to present the degree of curvature of the spine clearly for the doctor. In the example illustrated in FIG. 7, an assist line 85-4, which is an oblique line linking (passing through) two points that are the center of the ninth thoracic vertebra and the center of the femoral head, is an important assist line in terms of observation. Thus, the overall controller 50 adds other assist lines 85 that are parallel to and have the assist line 85-4 as a reference. The manner of adding the other assist lines 85 is optional. The other assist lines 85-1 to 85-3, 85-5, and 85-6 are optional, and do not need to be added. In cases in which these assist lines are added, the spacings between the parallel assist lines are preferably uniform spacings, and there is preferably an actual spacing of 3 cm to 5 cm therebetween to assist diagnosis and so on by the doctor.

In the example illustrated in FIG. 7, the overall controller 50 adds to the radiographic image 84 plural parallel assist lines 86 (86-1 to 86-13) that intersect (but are not orthogonal to in the example illustrated in FIG. 7) the assist lines 85. The assist lines 86 are similar to the assist lines 82 in Example 1 described above in that there is no particular limitation to the spacings between the respective assist lines 86.

In the example illustrated in FIG. 7, the overall controller 50 has also added a vertical line that passes through the center of the ninth thoracic vertebra as an assist line 87. However, the addition of the assist line 87 is optional.

Since the assist line 85-4 is an important assist line as described above, it may be displayed in a different color, with a different thickness, or the like to the other assist lines 85 and 86, so as to stand out.

Note that, in the above, a case in which the overall controller 50 adds the assist lines 85 and the assist lines 86 to the radiographic image 84 separately, has been explained. However, the assist line adding method is not limited thereto. For example, assist lines in a grid form formed of plural pre-combined lines may be superimposed on the radiographic image 84, similarly to as described in Example 1. In such a case, the overall controller 50 may rotate and add the predetermined grid form assist lines with the position of the assist line 85-4 as a reference.

Note that a case has been explained in the present example in which the interpreting purpose is for “adult spinal deformity” and the reference object is “ninth thoracic vertebra, femoral head”. Each of the control numbers 1-2 to 1-4, which have the same interpreting purpose but a different reference object, only have a different reference object from that in the present example, and so assist lines may be added by the overall controller 50 similarly to as described above, simply by changing the reference object from that in the present example.

### Example 3

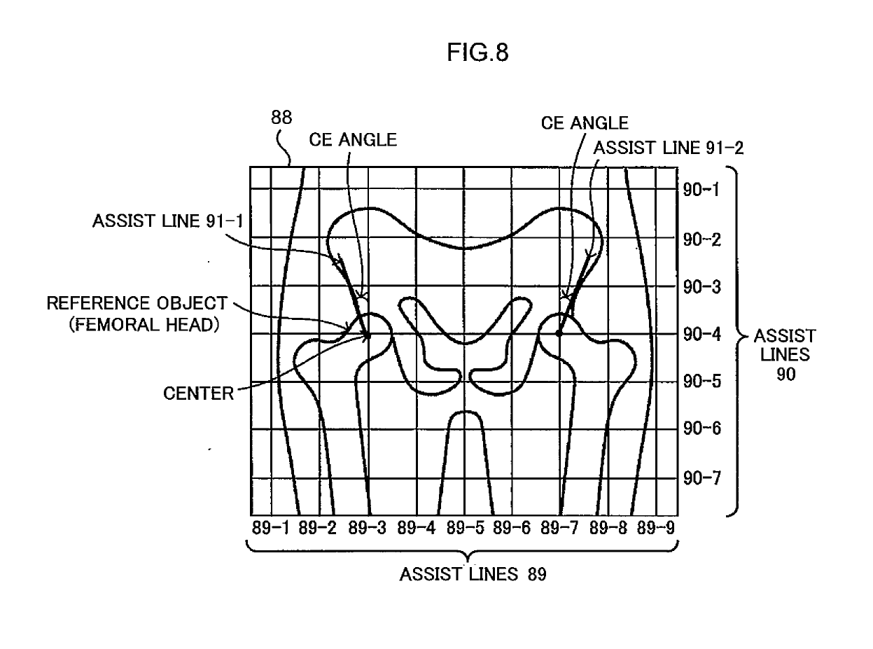

Explanation follows regarding a case in which the interpreting purpose is for “acetabular dysplasia”, and the reference object is “femoral head”. According to the assist line adding information 53 illustrated in the example in FIG. 3, in such a case, the control number is 2, the imaging type is “hip joint”, the assist line type is “vertical line”, and the assist line adding method is the method of the sub-program with the file name “***2”. FIG. 8 is an example of a schematic drawing in a case in which assist lines have been added to a radiographic image 88 obtained by imaging the hip joint of the imaging subject W in the present example.

The central edge angle (CE angle) generally serves as an index to diagnose acetabular dysplasia. A CE angle of 25° or less is a normal value. Note that, as illustrated in FIG. 8, the CE angle refers to an angle formed between a line (vertical line) running vertically from the center of the femoral head, and a line linking the center of the femoral head and the outer edge of the acetabular roof. Thus, in the present example, the overall controller 50 adds assist lines to the radiographic image in order to present the CE angle clearly for the doctor. In the example illustrated in FIG. 8, assist lines 89-3, 89-7, which are vertical lines passing through the center of each femoral head, are important, and so the assist lines 89-3, 89-7 are essential assist lines. The overall controller 50 accordingly adds other assist lines 89 that are parallel to and have the assist lines 89-3, 89-7 as a reference. Although the manner of adding the other assist lines 89 is optional, the assist line 89-5 is preferably added at the center of the body of the imaging subject W The other assist lines 89 (assist lines 89-1, 89-2, 89-4, 89-6, 89-8, and 89-9 in the example illustrated in FIG. 8) are optional, and do not need to be added. Similarly to the above-described Examples 1 and 2, in cases in which the assist lines 89 are added, the spacings between the parallel assist lines are preferably uniform spacings, and there is preferably an actual spacing of 3 cm to 5 cm therebetween to assist diagnosis and so on by the doctor.

In the example illustrated in FIG. 8, the overall controller 50 adds to the radiographic image 88 plural parallel assist lines 90 (90-1 to 90-7) that intersect (and are orthogonal to in the example illustrated in FIG. 8) the assist lines 89. The assist lines 90 are similar to the assist lines 82 in Example 1 and the assist lines 86 in Example 2 described above in that there is no particular limitation to the spacings between the respective assist lines 90.

In the example illustrated in FIG. 8, the overall controller 50 has also added lines linking the center of each femoral head and the respective outer edge of the acetabular roof as assist lines 91-1, 91-2; however, the addition of the assist lines 91-1, 91-2 is optional.

Note that the overall controller 50 may also derive the CE angle from the radiographic image 88 and display the derived CE angle on the display section 56.

### Example 4

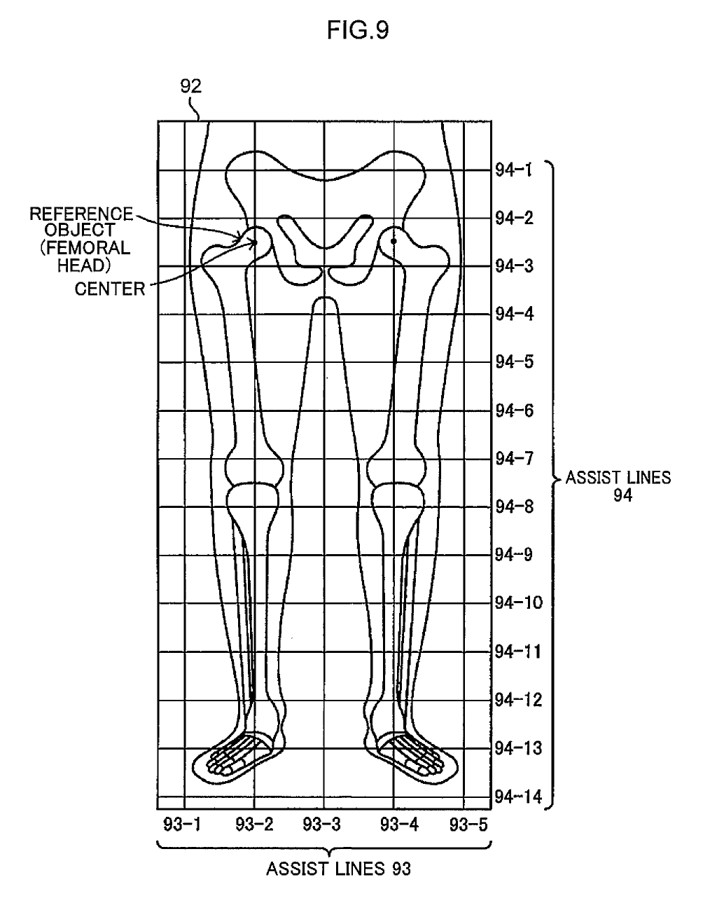

Explanation follows regarding a case in which the interpreting purpose is for “varus deformity of knee” or “malalignment”, and the reference object is “femoral head”. According to the assist line adding information 53 illustrated in the example in FIG. 3, in such a case, the control number is 3, the imaging type is “front of entire lower limbs”, the assist line type is “vertical line”, and the assist line adding method is the method of the sub-program with the file name “***3”. FIG. 9 is an example of a schematic drawing in a case in which assist lines have been added to a radiographic image 92 obtained by imaging the front of the entire lower limbs of the imaging subject W in the present example.

The degree of angulation of the tibia with respect to the axis of the femur, such as varus deformity of the knee, is generally observed to diagnose osteoarthritis of the knee. Thus, in the present example, the overall controller 50 adds assist lines to the radiographic image in order to present the degree of angulation of the tibia with respect to the axis of the femur clearly for the doctor. In the example illustrated in FIG. 9, assist lines 93-2, 93-4, which are vertical lines passing through the center of each femoral head, are important assist lines in terms of observation. The overall controller 50 accordingly adds other assist lines 93 that are parallel to and have the assist lines 93-2, 93-4 as a reference. The manner of adding the other assist lines 93 is optional. The other assist lines 93-1, 93-3, and 93-5 are optional, and do not need to be added. In cases in which these assist lines are added, the spacings between the parallel assist lines are preferably uniform spacings, and there is preferably an actual spacing of 3 cm to 5 cm therebetween to assist diagnosis and so on by the doctor.

In the example illustrated in FIG. 9, the overall controller 50 adds to the radiographic image 92 plural parallel assist lines 94 (94-1 to 94-14) that intersect (and, in the example illustrated in FIG. 9, are orthogonal to) the assist lines 93. The assist lines 94 are similar to the assist lines 82 in Example 1, the assist lines 86 in Example 2, and the assist lines 90 in Example 3 described above, in that there is no particular limitation to the spacings between the respective assist lines 94.

In the present example, a case has been explained in which the interpreting purpose is for “varus deformity of the knee” or “malalignment”. However, for example, the assist lines 93 and the assist lines 94 may be added similarly to in the present example in cases in which the interpreting purpose is for another form of osteoarthritis of the knee, such as valgus deformity of the knee.

### Example 5

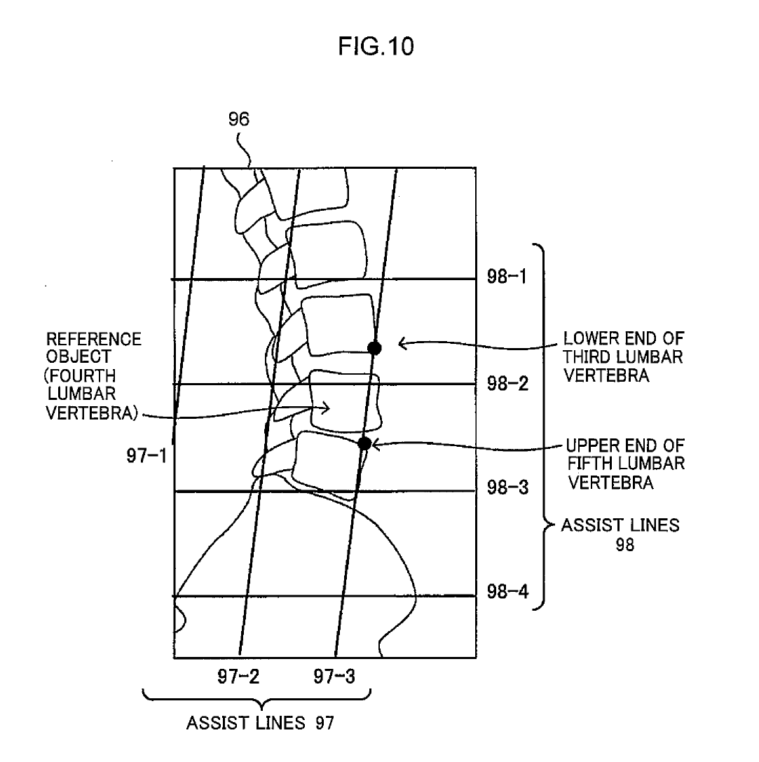

Explanation follows regarding a case in which the interpreting purpose is for “vertebral slippage”, and the reference object is “fourth lumbar vertebra”. According to the assist line adding information 53 illustrated in the example in FIG. 3, in such a case, the control numbers are 4-1 to 4-3. In the present example, explanation follows regarding a case in which assist lines are added according to control number 4-1 from out of these control numbers. In the case of control number 4-1, the imaging type is “side of entire spine or side of lumbar vertebrae”, the assist line type is “rectilinear”, and the assist line adding method is the method of the sub-program with the file name “***4-1”. FIG. 10 is an example of a schematic drawing in a case in which assist lines have been added to a radiographic image 96 obtained by imaging the side of the lumbar vertebrae of the imaging subject W in the present example.

Vertebral slippage generally refers to a lumbar vertebra slipping (becoming misaligned) toward the front or rear, is often seen in the fourth lumbar vertebra, and spondylolisthesis, in which a lumbar vertebra slides toward the front, is said to be more prevalent. The example illustrated in FIG. 10 illustrates a case in which the fourth lumbar vertebra is misaligned toward the front. The degree of misalignment of the misaligned lumbar vertebra (the fourth vertebra in FIG. 10) is observed to diagnose vertebral slippage. Thus, in the present example, the overall controller 50 adds assist lines to the radiographic image in order to present the degree of misalignment of the fourth lumbar vertebra clearly for the doctor.

In the example illustrated in FIG. 10, the fourth lumbar vertebra is the reference object, and an assist line 97-3, this being a straight line (oblique line) passing through a lower side end of an upper lumbar vertebra (third lumbar vertebra) and an upper side end of a lower lumbar vertebra (fifth lumbar vertebra), is an important assist line in terms of observation. The doctor is able to ascertain the degree of misalignment of the fourth lumbar vertebra by observing the position of the fourth lumbar vertebra with respect to the assist line 97-3. The overall controller 50 accordingly adds other assist lines 97 that are parallel to and have the assist line 97-3 as a reference. The manner of adding the other assist lines 97 is optional. The other assist lines 97-1, and 97-2 are optional, and do not need to be added. In cases in which these assist lines are added, the spacings between the parallel assist lines are preferably uniform spacings, and there is preferably an actual spacing of 3 cm to 5 cm therebetween, which is the same spacing as the size of representative lumbar vertebrae, to assist diagnosis and so on by the doctor.

In the example illustrated in FIG. 10, the overall controller 50 adds to the radiographic image 96 plural parallel assist lines 98 (98-1 to 98-4) that intersect (but are not orthogonal to in the example illustrated in FIG. 10) the assist lines 97. The assist lines 98 are similar to the assist lines 82 in Example 1, the assist lines 86 in Example 2, the assist lines 90 in Example 3, and the assist lines 94 in Example 4 described above in that there is no particular limitation to the spacings between the respective assist lines 98.

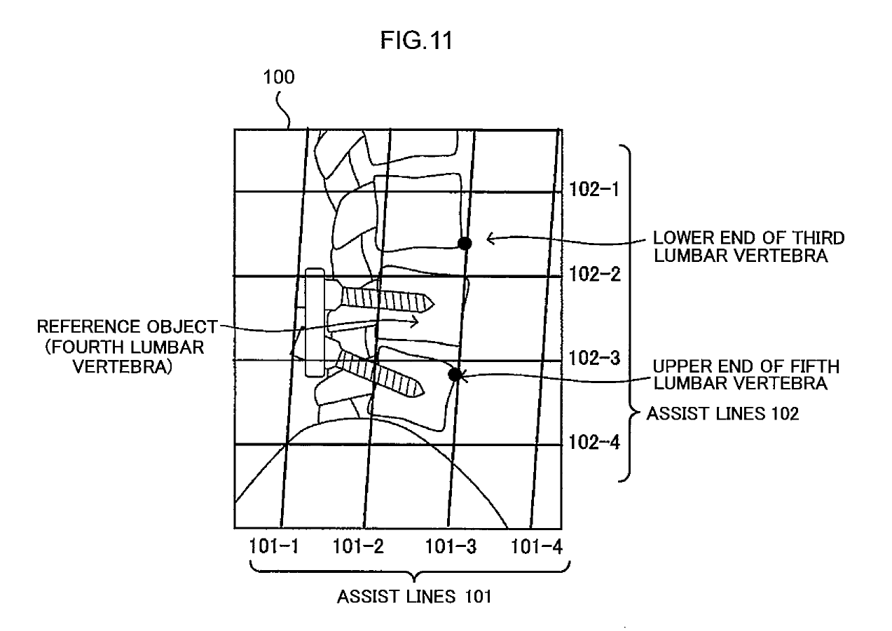

FIG. 11 is an example of a schematic drawing in a case in which assist lines have been added to a radiographic image 100 obtained by imaging the side of the lumbar vertebrae of the imaging subject W after the vertebral slippage has been operated on (spinal fusion surgery). Note that assist lines 101 (101-1 to 101-4) and assist lines 102 (102-1 to 102-4) illustrated in the example in FIG. 11 are added to the radiographic image 100 using a similar adding method to that of the assist lines 97 and 98 described above. For example, in cases in which affirmative determination is made at step S234 of the above-described image generation and display processing and the processing of steps S236 to S240 has been performed, the overall controller 50 displays on the display section 56 the radiographic image 96 illustrated in the example in FIG. 10 with the assist lines 97, 98 added, and the radiographic image 100 illustrated in the example in FIG. 11 with the assist lines 101, 102 added.

When the position (misalignment) of the fourth lumbar vertebra corresponding to the assist line 97-3 in the radiographic image 96 and the position (misalignment) of the fourth lumbar vertebra corresponding to the assist line 101-3 in the radiographic image 100 are compared, it is clear the radiographic image 100 has less misalignment. Namely, it is clear that the operation has suppressed the misalignment.

Note that the method of adding the assist lines 101 and 102 to the radiographic image 100 is not limited to that described above. For example, the spacing between the assist lines 101 and the spacing between the assist lines 97 may be made the same, the spacing between the assist lines 102 and the assist lines 98 may be made the same, or both may be made the same. The assist lines 97 and the assist lines 98 may also be added to the radiographic image 100 in order to assist observation of the change in the amount of misalignment of the fourth lumbar vertebra before and after the operation.

### Example 6

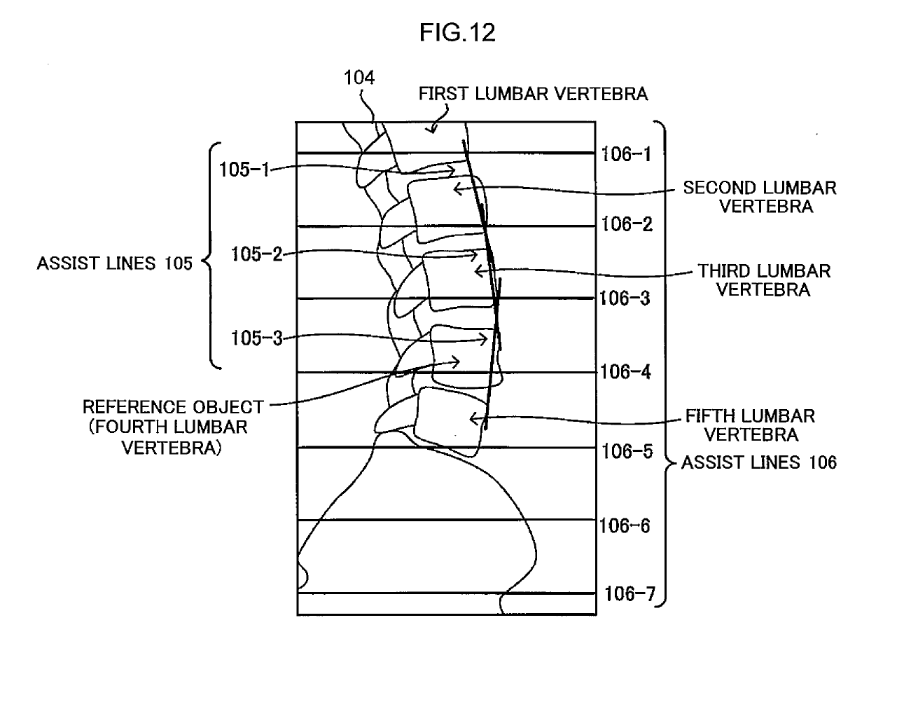

The present example explains regarding a case in which the interpreting purpose is for “vertebral slippage”, and the reference object is “fourth lumbar vertebra”, similarly to in Example 5. In the present example, explanation follows regarding a case in which assist lines are added according to the control number 4-2. In the case of control number 4-2, the imaging type is “side of entire spine or side of lumbar vertebrae”, the assist line type is “sectional straight line”, and the assist line adding method is the method of the sub-program with the file name “***4-2”. FIG. 12 is an example of a schematic drawing in a case in which assist lines have been added to a radiographic image 104 obtained by imaging the side of the lumbar vertebrae of the imaging subject W in the present example. Note that in the present example, the fourth lumbar vertebra corresponds to an example of an object of interest of the present disclosure.

In the example illustrated in FIG. 12, the fourth lumbar vertebra is the reference object, and rectilinear assist lines are added to every other of a predetermined number of lumbar vertebrae before and after the fourth lumbar vertebra. In the example illustrated in FIG. 12, a case is illustrated in which the overall controller 50 adds assist lines 105 to the radiographic image 104, these being an assist line 105-1 passing through a lower side end of the first lumbar vertebra and an upper side end of the third lumbar vertebra, an assist line 105-2 passing through a lower side end of the second lumbar vertebra and an upper side end of the fourth lumbar vertebra, and an assist line 105-3 passing through a lower side end of the third lumbar vertebra and an upper side end of the fourth lumbar vertebra. Although the overall controller 50 has also added assist lines 106 (106-1 to 106-7) to the radiographic image 104, the assist lines 106 are optional, and may for example be added similarly to those in Example 5.

Adding the assist lines 105 in this manner enables the degree of misalignment of the fourth lumbar vertebra to be compared to that of other lumbar vertebrae.

### Example 7

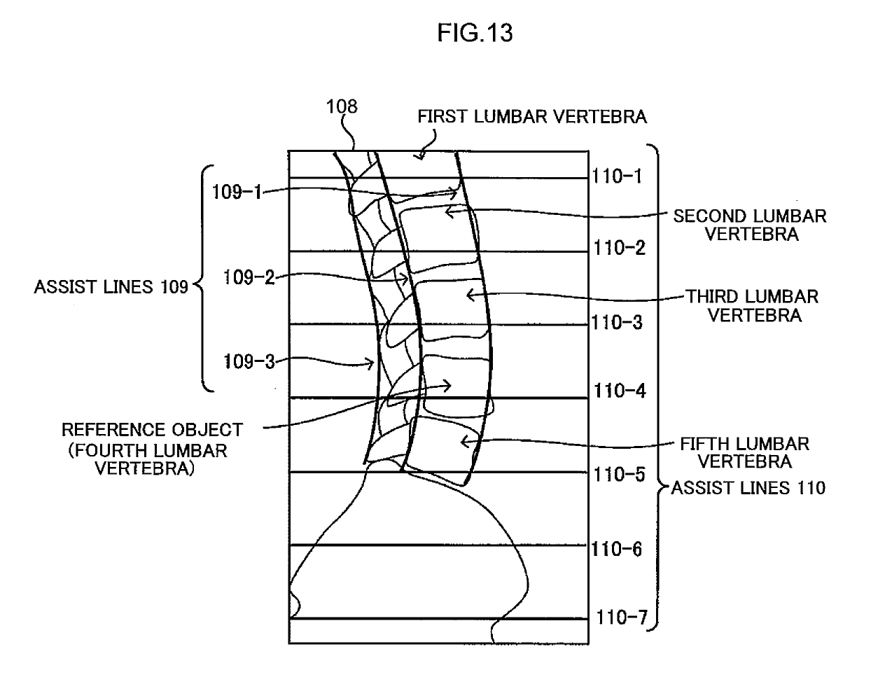

The present example explains regarding a case in which the interpreting purpose is for “vertebral slippage”, and the reference object is “fourth lumbar vertebra”, similarly to in Examples 5 and 6. In the present example, explanation follows regarding a case in which assist lines are added according to the control number 4-3. In the case of control number 4-3, the imaging type is “side of entire spine or side of lumbar vertebrae”, the assist line type is “curved line”, and the assist line adding method is the method of the sub-program with the file name “***4-3”. FIG. 13 is an example of a schematic drawing in a case in which assist lines have been added to a radiographic image 108 obtained by imaging the side of the lumbar vertebrae of the imaging subject W in the present example.

In the overall controller 50 of the present example, assist lines, which are curved lines joining the lumbar vertebrae other than the fourth lumbar vertebra, are added with the fourth lumbar vertebra as the reference object. The example illustrated in FIG. 13 illustrates a case in which assist lines 109 joining lower side ends of the lumbar vertebrae other than the fourth lumbar vertebra are added to the radiographic image 108. Specifically, the assist line 109-1 is an assist line linking the lower sides of the lumbar vertebra other than the fourth lumbar vertebra at right side ends thereof in a face-on view of the radiographic image 108, and the assist line 109-2 is an assist line linking the lower sides of the lumbar vertebra other than the fourth lumbar vertebra at left side ends thereof in the face-on view of the radiographic image 108. The assist line 109-3 is an assist line running along the spinal cord.

By thus adding the assist lines 109 based on excluding the fourth lumbar vertebra that has a large amount of misalignment, the degree of misalignment of the fourth lumbar vertebra can be ascertained by observing the position of the fourth lumbar vertebra with respect to the assist lines 109.

Although the overall controller 50 has also added assist lines 110 (110-1 to 110-7) to the radiographic image 108, the assist lines 110 are optional, and may for example be added similarly to those in Example 5.

Note that the method of adding assist lines by the overall controller 50 is not limited to those in Examples 1 to 7, and assist lines may be added by an adding method based on the interpreting purpose and the reference object. It is sufficient that the assist lines are useful in order for the doctor to interpret the radiographic image to make a diagnosis and so on according to each interpreting purpose (the kind of case, and so on), and there is no particular limitation to the specific kind of assist lines to be added.

For example, in cases in which the interpreting purpose is for “adult spinal deformity”, the reference object, the adding method, and so on are not limited to those in Examples 1 and 2. For example, the overall controller 50 may add to the radiographic image assist lines for clearly presenting to the doctor the Cobb angle, the front curvature angle of the lumbar vertebrae, the rear curvature angle of the thoracic vertebrae, and so on, used as an index to diagnose adult spinal deformity.

In cases in which the interpreting purpose is for “acetabular dysplasia”, the reference object, the adding method, and so on are not limited to those in Example 3. For example, the overall controller 50 may add to the radiographic image assist lines for clearly presenting the Sharp angle (the angle formed between a line connecting the outer edge of the acetabular roof and the acebutular teardrop together, and a line joining the left and right acetabular teardrops) used as an index to diagnose adult spinal deformity, and the like, to the doctor.

In cases in which the interpreting purpose indicates a kind of case, alignment issues, or the like relating to osteoarthritis of the knee, such as “varus deformity of the knee”, the reference object, the adding method, and so on are not limited to those in Example 4. For example, the overall controller 50 may add to the radiographic image assist lines for clearly presenting to the doctor the femoro tibial angle (FTA; an angle formed by the femur and the tibia), and so on, used as an index to diagnose osteoarthritis of the knee.

In cases in which the interpreting purpose is for “vertebral slippage”, the reference object, the adding method, and so on are not limited to those in Examples 5 to 7. For example, the assist line adding method may be differed according to whether they are added for spondylolisthesis, retrolisthesis, and spondylolysis. In Examples 5 to 7, the fourth lumbar vertebra is given as the reference object since vertebral slippage is often seen in the fourth lumbar vertebra; however, the reference object is not limited to the fourth lumbar vertebra. For example, another lumbar vertebra, such as the fifth lumbar vertebra, is sometimes misaligned. Thus, the reference object is not limited to the “fourth lumbar vertebra”, and may be “the most misaligned lumbar vertebra”. There is no particular limitation to the method of detecting the most misaligned lumbar vertebra from the radiographic image. For example, the most misaligned lumbar vertebra may be detected by performing processing on all the lumbar vertebrae to detect the misalignment amount of a single lumbar vertebra selected from the radiographic image with respect to a straight line passing through a lower end of the lumbar vertebra above the selected lumbar vertebra and through an upper end of the lumbar vertebra below the selected lumbar vertebra (see the assist line 97-3 in FIG. 10).

As explained above, the detection faces 19 of the plural radiation detectors 14 including the detection faces 19 that each detect radiation are disposed in an arranged state, and the overall controller 50 of the console 20 of the present exemplary embodiment acquires a radiographic image formed of stitched together radiographic images of the imaging subject imaged by the respective radiation detectors 14 of the radiographic imaging device 12 including the imaging face 35 with a wider range than a single detection face 19. The overall controller 50 also acquires information indicating the interpreting purpose. The overall controller 50 also adds to the radiographic image predetermined assist lines based on a method based on at least one from out of the reference object associated with the interpreting purpose in the acquired radiographic image, or the information indicating the interpreting purpose.

Thus, in the radiographic imaging system 10 of the present exemplary embodiment, due to the overall controller 50 of the console 20 adding assist lines to the radiographic image, there is no need for an actual plate for adding assist lines in order to imprint assist lines onto the radiographic image as a ghost image. Thus, the console 20 of the present exemplary embodiment may improve the work procedure of the operator, and may reduce the burden placed on the operator.

In the radiographic imaging system 10 of the present exemplary embodiment, the overall controller 50 performs stitching processing and step removal processing, since the radiographic imaging device 12 includes plural radiation detectors 14. If an actual plate for adding assist lines were employed to add assist lines, there would be a concern that artifacts might be generated during the image processing to the imprinted image of the plate for adding assist lines. For example, if a plate for adding assist lines were employed in order to add grid form assist lines, there would be a high possibility of artifacts being generated in the grid part thereof. In contrast thereto, there is no actual plate for adding assist lines employed in the console 20 of the present exemplary embodiment, and thus, there is no concern of such artifacts being generated.

Thus, in the console 20 of the radiographic imaging system 10 of the present exemplary embodiment, predetermined assist lines may be added to the radiographic image by an assist line adding method, based on the reference object corresponding to the interpreting purpose and the information indicating the interpreting purpose. This enables the assist lines for assisting interpretation to be displayed in a suitable position at the radiographic image.

Accordingly, the radiographic imaging system 10 of the present exemplary embodiment may add assist lines without employing an actual plate for adding assist lines, and may save the cost of a plate for adding assist lines.

In the present exemplary embodiment, the sub-program is treated as the assist line adding method, and the overall controller 50 executes the sub-program to add the assist lines to the radiographic image. This enables the assist line adding method to easily be updated as appropriate by updating the sub-programs in the console 20 of the present exemplary embodiment. Thus, the console 20 of the present exemplary embodiment may enable the assist lines to be added more appropriately.

Note that, in the radiographic imaging system 10 of the present exemplary embodiment, a case has been explained in which the overall controller 50 of the console 20 functions as each section of an image processing device of the present disclosure. However, there is no limitation to the present exemplary embodiment, and, for example, another device such as the image interpreting device 24 may include some or all of the functions of the respective sections of an image-processing device. Alternatively, for example, the device that performs the above-described image generation and display processing (see FIG. 5), and the display device that displays the radiographic image with the added assist lines may be different from each other. For example, the image generation and display processing (see FIG. 5) may be performed by the console 20, and the radiographic image with the added assist lines may be displayed by the image interpreting device 24.

Note that, in the present exemplary embodiment, a case in which the reference object is a bone, has been explained. However, the reference object is not limited to a bone. For example, the reference object may be another site on the imaging subject W, or may be a fastener such as a screw implanted inside the imaging subject W in an operation or the like.

In the radiation detector group 15 of the present exemplary embodiment, the radiation detector 14₁ is disposed nearest to the radiation irradiating device 16, and the radiation detector 14₃ is disposed furthest from the radiation irradiating device 16. However, the placement of the radiation detectors 14 is not limited to that in the present exemplary embodiment. For example, placement may be in what is referred to as a terraced shape in which the radiation detectors 14₁, 14₃ are disposed nearest to the radiation irradiating device 16, and the radiation detector 14₂ is disposed furthest from the radiation irradiating device 16. Alternatively, for example, placement may be in what is referred to as a terraced shape in which the radiation detectors 14₁, 14₃ are disposed furthest from the radiation irradiating device 16, and the radiation detector 14₂ is disposed nearest to the radiation irradiating device 16. In the radiation detector group 15 of the present exemplary embodiment, the end portions of the respective radiation detectors 14 are overlapped with each other. However, the respective radiation detectors 14 may be disposed in a state in which the detection faces 19 of the respective radiation detectors 14 are arranged in the same plane without the end portions overlapping each other. The radiation detectors 14 may also be disposed in a matrix shape, such as a 2 by 2 array.

In the present exemplary embodiment, a case in which plural radiation detectors 14 (the radiation detector group 15) are included inside one casing 13, has been explained. However, there is no limitation thereto. For example, each radiation detector 14 may be provided inside its own individual casing.

There is no particular limitation to the type of radiation R of the present exemplary embodiment, and X-rays or y rays, for example, may be applied.

Configurations of the radiographic imaging system 10, the radiation detector group 15, the radiation detectors 14, the console 20, etc. explained in the present exemplary embodiment are merely examples thereof, and obviously modifications may be implemented depending on the circumstances within a range not departing from the spirit of the present disclosure.

What is claimed is:

1. An image processing device comprising:
a radiographic image acquisition section that acquires a radiographic image, the radiographic image generated by stitching together radiographic images of an imaging subject imaged by a plurality of radiation detectors, each of which includes a detection face for detecting radiation;
a purpose acquisition section that acquires information indicating an interpreting purpose; and
an adding section that adds a predetermined assist line to the radiographic image, on the basis of at least one of a reference object corresponding to the interpreting purpose in the radiographic image acquired by the radiographic image acquisition section or the information indicating the interpreting purpose.

2. The image processing device of claim 1, wherein at least one type of the reference object is predetermined for each interpreting purpose.

3. The image processing device of claim 1, wherein:
the reference object is a bone of the imaging subject; and
the adding section extracts an image of the reference object from the radiographic image, and adds to the radiographic image an assist line, on the basis of at least one of an image of the reference object or the information indicating the interpreting purpose.

4. The image processing device of claim 1, further comprising a receiving section that receives designation of the reference object,
wherein, in cases in which the receiving section has received designation of the reference object, the adding section adds to the radiographic image an assist line, on the basis of at least one of the reference object corresponding to the received designation or the information indicating the interpreting purpose.

5. The image processing device of claim 1, wherein, in cases in which the assist lines are assist lines in which a plurality of lines are combined in grid form, the adding section rotates the grid form assist lines by an angle derived using the reference object corresponding to the interpreting purpose and the information indicating the interpreting purpose, and adds the rotated assist lines to the radiographic image.

6. The image processing device of claim 1, wherein, in cases in which an assist line is added to a plurality of radiographic images, the adding section uses the same basis as the basis for adding the assist line to the one radiographic image to add an assist line to another radiographic image from out of the plurality of radiographic images.

7. The image processing device of claim 1, wherein the adding section only adds the assist line to an image of an object of interest determined by the interpreting purpose in the radiographic image.

8. A radiographic imaging system comprising:
a radiographic imaging device that includes a plurality of radiation detectors disposed such that detection faces of the plurality of radiation detectors are in an arranged state, and includes an imaging face having a wider range than one of the detection faces; and
the image processing device of claim 1, which adds an image of an assist line to a radiographic image imaged by the radiographic imaging device.

9. The radiographic imaging system of claim 8, further comprising a display device that displays the radiographic image to which the image of the assist line has been added by the image processing device.

10. An image processing method comprising:
acquiring a radiographic image and information indicating an interpreting purpose, the radiographic image generated by stitching together radiographic images of an imaging subject imaged by a plurality of radiation detectors, each of which includes a detection face for detecting radiation; and
adding a predetermined assist line to the radiographic image based on at least one of a reference object corresponding to the interpreting purpose in the acquired radiographic image or the information indicating the interpreting purpose.

11. A non-transitory computer readable medium storing a program causing a computer to execute a process for image processing, the process comprising:
acquiring a radiographic image and information indicating an interpreting purpose, the radiographic image generated by stitching together radiographic images of an imaging subject imaged by a plurality of radiation detectors, each of which includes a detection face for detecting radiation; and
adding a predetermined assist line to the radiographic image, based on at least one of a reference object corresponding to the interpreting purpose in the acquired radiographic image or the information indicating the interpreting purpose.
